(19) United States
(12) Patent Application Publication
(10) Pub. No.: US 2024/0415473 A1
(43) Pub. Date: Dec. 19, 2024

(54) INFORMATION PROCESSING APPARATUS, INFORMATION PROCESSING METHOD, INFORMATION PROCESSING PROGRAM, LEARNING DEVICE, LEARNING METHOD, LEARNING PROGRAM, AND DISCRIMINATIVE MODEL

(71) Applicants: KYOTO UNIVERSITY, Kyoto-shi (JP); FUJIFILM CORPORATION, Tokyo (JP)

(72) Inventors: Akira ISHII, Kyoto-shi (JP); Hidehisa NISHI, Kyoto-shi (JP); Takuya FUCHIGAMI, Tokyo (JP)

(21) Appl. No.: 18/817,161

(22) Filed: Aug. 27, 2024

Related U.S. Application Data
(63) Continuation of application No. PCT/JP2022/041924, filed on Nov. 10, 2022.

(30) Foreign Application Priority Data
Mar. 7, 2022 (JP) 2022-034781

(51) Int. Cl. A61B 6/03; G06T 7/00

(52) U.S. Cl. CPC A61B 6/03; G06T 7/0012; G06T 2207/10081; G06T 2207/30016

(57) ABSTRACT

A processor acquires at least one of first information representing any one of an infarction region or a large vessel occlusion region in a non-contrast CT image of a head of a patient, information representing an anatomical region of a brain, or clinical information, acquires second information representing a candidate of the other of the infarction region or the large vessel occlusion region in the non-contrast CT image, and derives third information representing the other of the infarction region or the large vessel occlusion region in the non-contrast CT image based on at least one of the first information, the information representing the anatomical region of the brain, or the clinical information, and the second information.

### CROSS-REFERENCE TO RELATED APPLICATIONS

This application is a continuation of International Application No. PCT/JP2022/041924, filed on Nov. 10, 2022, which claims priority from Japanese Patent Application No. 2022-034781, filed on Mar. 7, 2022. The entire disclosure of each of the above applications is incorporated herein by reference.

### BACKGROUND

### Technical Field

The present disclosure relates to an information processing apparatus, an information processing method, an information processing program, a learning device, a learning method, a learning program, and a discriminative model.

### Related Art

In recent years, with the progress of medical devices, such as a computed tomography (CT) apparatus and a magnetic resonance imaging (MRI) apparatus, an image diagnosis can be made by using a medical image having a higher quality and a higher resolution. In particular, in a case in which a target part is the brain, it is possible to specify a region in which a blood vessel disorder of the brain, such as a cerebral infarction or a cerebral hemorrhage, occurs by the image diagnosis using a CT image, an MRI image, or the like. Therefore, various methods for supporting the image diagnosis have been proposed.

By the way, the cerebral infarction is a disease in which a brain tissue is damaged by occlusion of a cerebral blood vessel, and is known to have a poor prognosis. In a case in which the cerebral infarction is developed, irreversible cell death progresses with the elapse of time. Therefore, how to shorten the time to the start of treatment has become an important issue. Here, in the application of thrombectomy treatment method, which is a typical treatment method for the cerebral infarction, two pieces of information, “degree of extent of infarction” and “presence or absence of large vessel occlusion (LVO)”, are required (see Appropriate Use Guidelines For Percutaneous Transluminal Cerebral Thrombectomy Devices, 4th edition, March 2020, p. 12-(1)).

On the other hand, in the diagnosis of a patient suspected of having a brain disease, the presence or absence of bleeding in the brain is often confirmed before confirming the cerebral infarction. Since bleeding in the brain can be clearly confirmed on a non-contrast CT image, a diagnosis using the non-contrast CT image is first made for the patient suspected of having the brain disease. However, in the non-contrast CT image, a difference in pixel value between a region of the cerebral infarction and the other region is not so large. Moreover, in the non-contrast CT image, a hyperdense artery sign (HAS) reflecting a thrombus that causes the large vessel occlusion can be visually recognized, but is not clear, so that it is difficult to specify a large vessel occlusion region. As described above, it is often difficult to specify an infarction region and the large vessel occlusion region by using the non-contrast CT image. Therefore, after the diagnosis using the non-contrast CT image, the MRI image or a contrast CT image is acquired to diagnose whether or not the cerebral infarction has developed, specify the large vessel occlusion region, and confirm the degree of extent of the infarction in a case in which the cerebral infarction has occurred.

However, in a case in which whether or not the cerebral infarction has developed is diagnosed by acquiring the MRI image and the contrast CT image after the diagnosis using the CT image, the elapsed time from the development of the infarction is long and the start of treatment is delayed, as a result, there is a high probability that the prognosis will be poor.

Therefore, a method for automatically extracting an infarction region and the large vessel occlusion region from the non-contrast CT image has been proposed. For example, JP2020-054580A proposes a method of specifying an infarction region and a thrombus region by using a discriminator that has been trained to extract the infarction region from a non-contrast CT image and a discriminator that has been trained to extract the thrombus region from the non-contrast CT image.

On the other hand, an appearance place of HAS representing the large vessel occlusion region is changed depending on which blood vessel is occluded, and an appearance varies depending on an angle of a tomographic plane with respect to the brain in the CT image, a property of a thrombus, a degree of occlusion, and the like. Moreover, it may be difficult to distinguish from similar structures in the vicinity, such as calcification. Moreover, the infarction region is generated in a blood vessel dominant region by the blood vessel in which the HAS is generated. Therefore, in a case in which the large vessel occlusion region can be specified, it is easy to specify the infarction region.

### SUMMARY OF THE INVENTION

The present disclosure has been made in view of the above circumstances, and an object of the present disclosure is to enable accurate specification of a large vessel occlusion region or an infarction region by using a non-contrast CT image of a head.

The present disclosure relates to an information processing apparatus comprising: at least one processor, in which the processor acquires at least one of first information representing any one of an infarction region or a large vessel occlusion region in a non-contrast CT image of a head of a patient, information representing an anatomical region of a brain, or clinical information, acquires second information representing a candidate of the other of the infarction region or the large vessel occlusion region in the non-contrast CT image, and derives third information representing the other of the infarction region or the large vessel occlusion region in the non-contrast CT image based on at least one of the first information, the information representing the anatomical region of the brain, or the clinical information, and the second information.

It should be noted that, in the information processing apparatus according to the present disclosure, the processor may further acquire the non-contrast CT image, and may derive the third information further based on the non-contrast CT image.

In addition, in the information processing apparatus according to the present disclosure, the processor may derive the third information by using a discriminative model that has been trained to output the third information in a case in which at least one of the first information, the information representing the anatomical region of the brain, or the clinical information, the non-contrast CT image, and the second information are input.

In addition, in the information processing apparatus according to the present disclosure, the processor may derive the third information further based on information on symmetrical regions with respect to a midline of the brain in at least the non-contrast CT image out of the first information, the non-contrast CT image, and the second information.

In addition, in the information processing apparatus according to the present disclosure, the information on the symmetrical regions may be inversion information obtained by inverting at least the non-contrast CT image out of the first information, the non-contrast CT image, and the second information, with respect to the midline of the brain.

In addition, in the information processing apparatus according to the present disclosure, the processor may acquire the first information by extracting any one of the infarction region or the large vessel occlusion region from the non-contrast CT image, and may acquire the second information by extracting the candidate of the other of the infarction region or the large vessel occlusion region from the non-contrast CT image.

In addition, in the information processing apparatus according to the present disclosure, the processor may derive quantitative information for at least one of the first information, the second information, or the third information, and may display the quantitative information.

The present disclosure relates to a learning device comprising: at least one processor, in which the processor acquires i) a non-contrast CT image of a head of a patient with cerebral infarction, ii) at least one of first information representing any one of an infarction region or a large vessel occlusion region in the non-contrast CT image, information representing an anatomical region of a brain, or clinical information, and iii) training data including input data consisting of second information representing a candidate of the other of the infarction region or the large vessel occlusion region in the non-contrast CT image, and correct answer data consisting of third information representing the other of the infarction region and the large vessel occlusion region in the non-contrast CT image, and trains a neural network through machine learning using the training data to construct a discriminative model that outputs the third information in a case in which at least one of the first information, the information representing the anatomical region of the brain, or the clinical information, the non-contrast CT image, and the second information are input.

The present disclosure relates to a discriminative model that, in a case in which i) a non-contrast CT image of a head of a patient, ii) at least one of first information representing any one of an infarction region or a large vessel occlusion region in the non-contrast CT image, information representing an anatomical region of a brain, or clinical information, and iii) second information representing a candidate of the other of the infarction region or the large vessel occlusion region in the non-contrast CT image are input, outputs third information representing the other of the infarction region or the large vessel occlusion region in the non-contrast CT image.

The present disclosure relates to an information processing method comprising: acquiring at least one of first information representing any one of an infarction region or a large vessel occlusion region in a non-contrast CT image of a head of a patient, information representing an anatomical region of a brain, or clinical information; acquiring second information representing a candidate of the other of the infarction region or the large vessel occlusion region in the non-contrast CT image; and deriving third information representing the other of the infarction region or the large vessel occlusion region in the non-contrast CT image based on at least one of the first information, the information representing the anatomical region of the brain, or the clinical information, and the second information.

The present disclosure relates to a learning method comprising: acquiring i) a non-contrast CT image of a head of a patient with cerebral infarction, ii) at least one of first information representing any one of an infarction region or a large vessel occlusion region in the non-contrast CT image, information representing an anatomical region of a brain, or clinical information, and iii) training data including input data consisting of second information representing a candidate of the other of the infarction region or the large vessel occlusion region in the non-contrast CT image, and correct answer data consisting of third information representing the other of the infarction region and the large vessel occlusion region in the non-contrast CT image; and training a neural network through machine learning using the training data to construct a discriminative model that outputs the third information in a case in which at least one of the first information, the information representing the anatomical region of the brain, or the clinical information, the non-contrast CT image, and the second information are input.

It should be noted that programs casing a computer to execute the information processing method and the learning method according to the present disclosure may be provided.

According to the present disclosure, the large vessel occlusion region or the infarction region can be accurately specified by using the non-contrast CT image of the head.

### BRIEF DESCRIPTION OF THE DRAWINGS

FIG. 1 is a diagram showing a schematic configuration of a medical information system to which an information processing apparatus and a learning device according to a first embodiment of the present disclosure are applied.

FIG. 2 is a diagram showing a schematic configuration of the information processing apparatus and the learning device according to the first embodiment.

FIG. 3 is a functional configuration diagram of the information processing apparatus and the learning device according to the first embodiment.

FIG. 4 is a schematic block diagram showing a configuration of an information derivation unit in the first embodiment.

FIG. 5 is a diagram schematically showing a configuration of a U-Net.

FIG. 6 is a diagram showing inversion of a feature amount map.

FIG. 7 is a diagram showing training data for training the U-Net corresponding to a third discriminative model in the first embodiment.

FIG. 8 is a diagram showing an artery and a dominant region in a brain.

FIG. 9 is a diagram showing a display screen.

FIG. 10 is a flowchart showing learning processing performed in the first embodiment.

FIG. 11 is a flowchart showing information processing performed in the first embodiment.

FIG. 12 is a schematic block diagram showing a configuration of the information derivation unit in the first embodiment.

FIG. 13 is a diagram showing training data for training a U-Net corresponding to a third discriminative model in a second embodiment.

FIG. 14 is a flowchart showing learning processing performed in the second embodiment.

FIG. 15 is a flowchart showing information processing performed in the second embodiment.

FIG. 16 is a schematic block diagram showing a configuration of an information derivation unit in a third embodiment.

FIG. 17 is a diagram showing training data for training a U-Net corresponding to a third discriminative model in the third embodiment.

### DETAILED DESCRIPTION

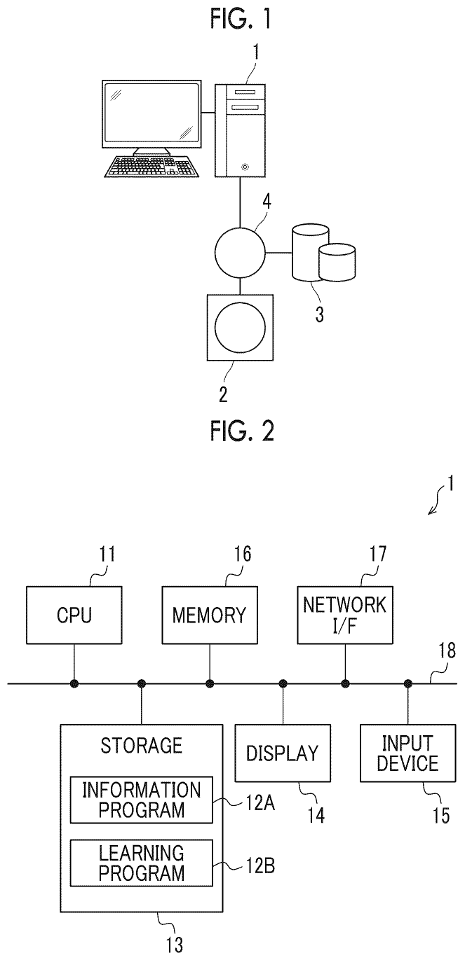

In the following, a first embodiment of the present disclosure will be described with reference to the drawings. FIG. 1 is a hardware configuration diagram showing an outline of a diagnosis support system to which an information processing apparatus and a learning device according to the first embodiment of the present disclosure are applied. As shown in FIG. 1, in the diagnosis support system, an information processing apparatus 1, a three-dimensional image capturing apparatus 2, and an image storage server 3 according to the first embodiment are connected to each other in a communicable state via a network 4. It should be noted that the information processing apparatus 1 includes the learning device according to the present embodiment.

The three-dimensional image capturing apparatus 2 is an apparatus that images a diagnosis target part of a subject to generate a three-dimensional image representing the part, and is, specifically, a CT apparatus, an MRI apparatus, a PET apparatus, and the like. A medical image generated by the three-dimensional image capturing apparatus 2 is transmitted to and stored in the image storage server 3. It should be noted that, in the present embodiment, the diagnosis target part of a patient who is the subject is the brain, the three-dimensional image capturing apparatus 2 is the CT apparatus, and a three-dimensional CT image G0 of the head of the patient who is the subject is generated in the CT apparatus. It should be noted that, in the present embodiment, the CT image G0 is a non-contrast CT image acquired by performing imaging without using a contrast agent.

The image storage server 3 is a computer that stores and manages various data, and comprises a large-capacity external storage device and software for database management. The image storage server 3 communicates with another device via the wired or wireless network 4 to transmit and receive image data and the like to and from the other device. Specifically, the image storage server 3 acquires various data including the image data of the CT image generated by the three-dimensional image capturing apparatus 2 via the network, and stores and manages the data in a recording medium, such as the large-capacity external storage device. Moreover, training data for constructing a discriminative model is also stored in the image storage server 3, as will be described below. It should be noted that a storage format of the image data and the communication between the devices via the network 4 are based on a protocol, such as digital imaging and communication in medicine (DICOM).

Next, the information processing apparatus and the learning device according to the first embodiment of the present disclosure will be described. FIG. 2 shows a hardware configuration of the information processing apparatus and the learning device according to the first embodiment. As shown in FIG. 2, the information processing apparatus and the learning device (hereinafter, represented by the information processing apparatus) 1 includes a central processing unit (CPU) 11, a non-volatile storage 13, and a memory 16 as a transitory storage area. Moreover, the information processing apparatus 1 includes a display 14, such as a liquid crystal display, an input device 15, such as a keyboard and a mouse, and a network interface (I/F) 17 connected to the network 4. The CPU 11, the storage 13, the display 14, the input device 15, the memory 16, and the network I/F 17 are connected to a bus 18. It should be noted that the CPU 11 is an example of a processor according to the present disclosure.

The storage 13 is realized by a hard disk drive (HDD), a solid state drive (SSD), a flash memory, or the like. An information processing program 12A and a learning program 12B are stored in the storage 13 as a storage medium. The CPU 11 reads out the information processing program 12A and the learning program 12B from the storage 13, loads the information processing program 12A and the learning program 12B in the memory 16, and executes the loaded information processing program 12A and learning program 12B.

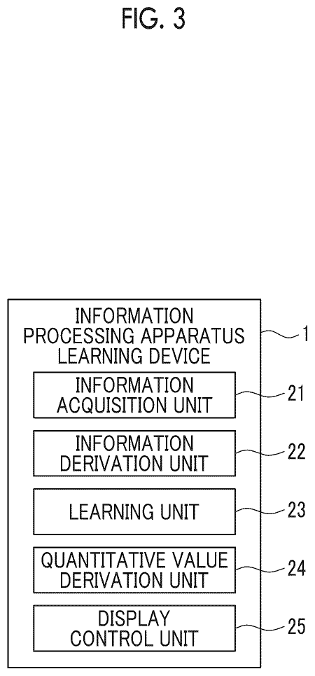

Next, a functional configuration of the information processing apparatus according to the first embodiment will be described. FIG. 3 is a diagram showing the functional configuration of the information processing apparatus according to the first embodiment. As shown in FIG. 3, the information processing apparatus 1 comprises an information acquisition unit 21, an information derivation unit 22, a learning unit 23, a quantitative value derivation unit 24, and a display control unit 25. Then, by executing the information processing program 12A, the CPU 11 functions as the information acquisition unit 21, the information derivation unit 22, the quantitative value derivation unit 24, and the display control unit 25. Moreover, the CPU 11 functions as the learning unit 23 by executing the learning program 12B.

The information acquisition unit 21 acquires the non-contrast CT image G0 of the head of the patient from the image storage server 3. Moreover, the information acquisition unit 21 acquires the input data for training a neural network from the image storage server 3 in order to construct the discriminative model described below.

The information acquisition unit 21 acquires the non-contrast CT image G0 of the head of the patient from the image storage server 3. Moreover, the information acquisition unit 21 acquires the input data for training a neural network from the image storage server 3 in order to construct the discriminative model described below.

The information derivation unit 22 acquires at least one of first information representing any one of an infarction region or a large vessel occlusion region in a CT image G0, information representing an anatomical region of a brain, or clinical information, acquires second information representing a candidate of the other of the infarction region or the large vessel occlusion region in the CT image G0, and derives third information representing the other of the infarction region or the large vessel occlusion region in the CT image G0 based on at least one of the CT image G0, the first information, the information representing the anatomical region of the brain, or the clinical information, and the second information. In the present embodiment, the first information representing the infarction region in the CT image G0 is acquired, the second information representing the candidate of the large vessel occlusion region in the CT image G0 is acquired, and the third information representing the large vessel occlusion region in the CT image G0 is derived based on the CT image G0, the first information, and the second information.

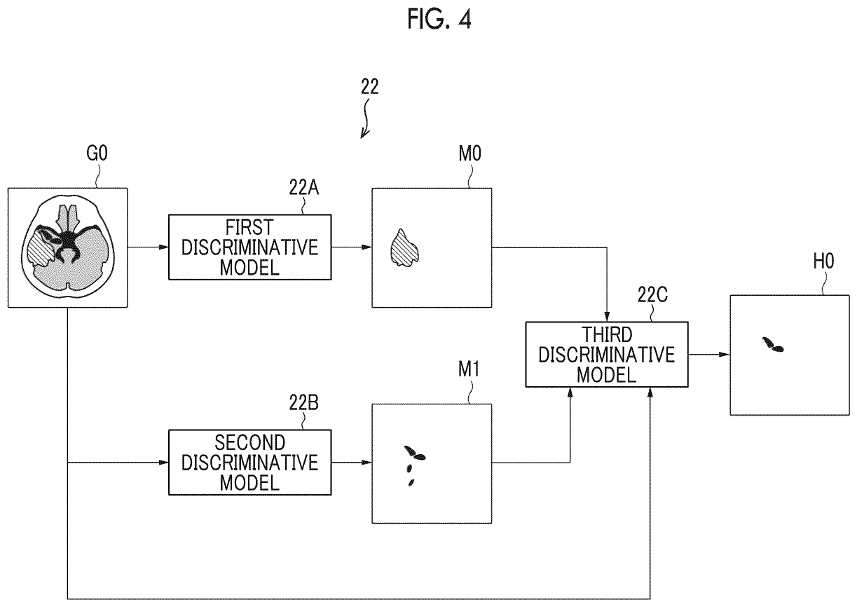

FIG. 4 is a schematic block diagram showing a configuration of the information derivation unit in the first embodiment. As shown in FIG. 4, the information derivation unit 22 includes a first discriminative model 22A, a second discriminative model 22B, and a third discriminative model 22C. The first discriminative model 22A is constructed by training a convolutional neural network (CNN) through machine learning to extract the infarction region of the brain, as the first information, from the CT image G0 which is a processing target. For the construction of the first discriminative model 22A, for example, the method disclosed in JP2020-054580A can be used. Specifically, the first discriminative model 22A can be constructed by training the CNN through machine learning using, as the training data, the non-contrast CT image of the head and a mask image representing the infarction region in the non-contrast CT image. As a result, the first discriminative model 22A extracts the infarction region in the CT image G0 from the CT image G0 to output a mask image M0 representing the infarction region in the CT image G0.

The second discriminative model 22B is constructed by training the CNN through machine learning to extract the candidate of the large vessel occlusion region, as the second information, from the CT image G0 which is a processing target. For the construction of the second discriminative model 22B, for example, the method disclosed in JP2020-054580A can be used. Specifically, the second discriminative model 22B can be constructed by training the CNN through machine learning using, as the training data, the non-contrast CT image of the head and the mask image representing the large vessel occlusion region in the non-contrast CT image. As a result, the second discriminative model 22B extracts the large vessel occlusion region in the CT image G0 from the CT image G0, to output a mask image M1 representing the large vessel occlusion region in the CT image G0. It should be noted that, in the first embodiment, both the second discriminative model 22B and the third discriminative model 22C extract the large vessel occlusion region from the CT image G0, but the large vessel occlusion region extracted by the second discriminative model 22B is used as a large vessel occlusion region candidate. The second discriminative model 22B may be a CNN that gives importance to the sensitivity. In addition, a model that extracts the large vessel occlusion region candidate by, for example, threshold value processing may be used as the second discriminative model 22B, instead of the model constructed by machine learning such as the CNN.

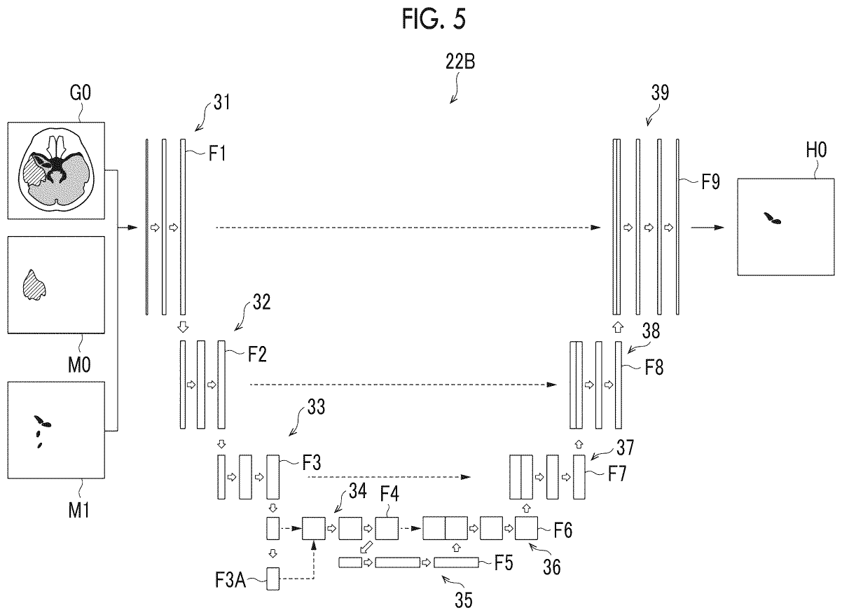

The third discriminative model 22C is constructed by training the U-Net, which is one type of the convolutional neural network, through machine learning using a large amount of the training data, to extract the large vessel occlusion region in the CT image G0 as the third information based on the CT image G0, the mask image M0 representing the infarction region in the CT image G0, and the mask image M1 representing the large vessel occlusion region candidate in the CT image G0. FIG. 5 is a diagram schematically showing a configuration of the U-Net. As shown in FIG. 5, the third discriminative model 22C is configured by 9 layers of a first layer 31 to a ninth layer 39. It should be noted that, in the present embodiment, in a case in which the third information is derived, information on the symmetrical regions with respect to the midline of the brain in at least the CT image G0 out of the CT image G0, the mask image M0 representing the infarction region, and the mask image M1 representing the large vessel occlusion region candidate is used. The information on the symmetrical regions with respect to the midline of the brain will be described below.

In the present embodiment, the CT image G0, the mask image M0 representing the infarction region in the CT image G0, and the mask image M1 representing the large vessel occlusion region candidate in the CT image G0 are input in combination to the first layer 31. It should be noted that, depending on the CT image G0, there is a case in which the midline of the brain is inclined with respect to a perpendicular bisector of the CT image G0 in the image. In such a case, the brain in the CT image G0 is rotated such that the midline of the brain matches the perpendicular bisector of the CT image G0. In this case, it is required to perform the same rotation processing on the mask image M0 and the mask image M1.

The first layer 31 includes two convolutional layers, and outputs a feature amount map F1 in which three feature amount maps after the convolution of the CT image G0, the mask image M0, and the mask image M1 are integrated. The integrated feature amount map F1 is input to the ninth layer 39 as shown by a broken line in FIG. 5. Moreover, the integrated feature amount map F1 is subjected to pooling, is reduced in size to ½, and is input to the second layer 32. In FIG. 5, the pooling is indicated by a downward arrow. It is assumed that, in a case of the convolution, for example, a 3×3 kernel is used in the present embodiment, but the present disclosure is not limited to this. Moreover, it is assumed that, in the pooling, the maximum value of the four pixels is adopted, but the present disclosure is not limited to this.

The second layer 32 includes two convolutional layers, and a feature amount map F2 output from the second layer 32 is input to the eighth layer 38 as shown by a broken line in FIG. 5. Moreover, the feature amount map F2 is subjected to pooling, is reduced in size to ½, and is input to the third layer 33.

The third layer 33 also includes two convolutional layers, and a feature amount map F3 output from the third layer 33 is input to the seventh layer 37 as shown by a broken line in FIG. 5. Moreover, the feature amount map F3 is subjected to pooling, is reduced in size to ½, and is input to the fourth layer 34.

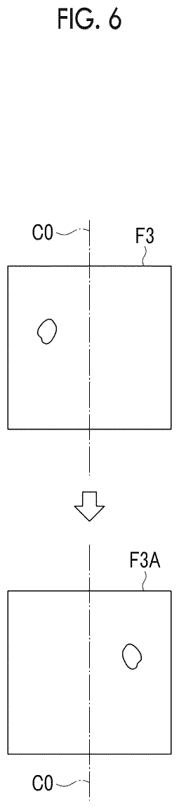

In addition, in the present embodiment, in a case in which the third information is derived, the information on the symmetrical regions with respect to the midline of the brain in the CT image G0, the mask image M0 representing the infarction region of the CT image G0, and the mask image M1 representing the large vessel occlusion region candidate is used. Therefore, in the third layer 33 of the third discriminative model 22C, the feature amount map F3 subjected to the pooling is inverted left and right with respect to the midline of the brain, and an inversion feature amount map F3A is derived. FIG. 6 is a diagram showing the inversion of the feature amount map. As shown in FIG. 6, the feature amount map F3 is inverted left and right with respect to a midline CO of the brain, and the inversion feature amount map F3A is derived. The inversion feature amount map F3A is an example of inversion information in the present disclosure. It should be noted that, in the present embodiment, the inversion information is generated inside the U-Net. However, at a point in time at which the CT image G0 and the mask image M0 are input to the first layer 31, the inversion image of at least the CT image G0 out of the CT image G0 and the mask images M0 and M1 may be generated, the CT image G0, the inversion image of the CT image G0, the mask image M0, and the mask image M1 are input in combination to the first layer 31. The inversion image of at least one of the mask image M0 or the mask image M1 may be generated in addition to the inversion image of the CT image G0. For example, in a case in which the inversion images of both the mask image M0 and the mask image M1 are generated, the CT image G0, the inversion image of the CT image G0, the mask image M0, the mask image M1, the inversion image of the mask image M0, and the inversion image of the mask image M1 may be input in combination to the first layer 31. In this case, the inversion image need only be generated by rotating the brain in the CT image G0 or rotating the mask in the mask images M0 and M1 such that the midline of the brain matches the perpendicular bisector of the CT image G0 and the mask images M0 and M1.

The fourth layer 34 also includes two convolutional layers, and the feature amount map F3 subjected to the pooling and the inversion feature amount map F3A are input to the first convolutional layer. A feature amount map F4 output from the fourth layer 34 is input to the sixth layer 36 as shown by a broken line in FIG. 5. Moreover, the feature amount map F4 is subjected to pooling, is reduced in size to ½, and is input to the fifth layer 35.

The fifth layer 35 includes one convolutional layer, and a feature amount map F5 output from the fifth layer 35 is subjected to upsampling, is doubled in size, and is input to the sixth layer 36. In FIG. 5, the upsampling is indicated by an upward arrow.

The sixth layer 36 includes two convolutional layers, and performs a convolution operation by integrating the feature amount map F4 from the fourth layer 34 and the feature amount map F5, which is subjected to the upsampling, from the fifth layer 35. A feature amount map F6 output from the sixth layer 36 is subjected to upsampling, is doubled in size, and is input to the seventh layer 37.

The seventh layer 37 includes two convolutional layers, and performs the convolution operation by integrating the feature amount map F3 from the third layer 33 and the feature amount map F6, which is subjected to the upsampling, from the sixth layer 36. A feature amount map F7 output from the seventh layer 37 is subjected to upsampling and is input to the eighth layer 38.

The eighth layer 38 includes two convolutional layers, and performs the convolution operation by integrating the feature amount map F2 from the second layer 32 and the feature amount map F7, which is subjected to the upsampling, from the seventh layer 37. A feature amount map output from the eighth layer 38 is subjected to upsampling and is input to the ninth layer 39.

The ninth layer 39 includes three convolutional layers, and performs the convolution operation by integrating the feature amount map F1 from the first layer and the feature amount map F8, which is subjected to the upsampling, from the eighth layer 38. A feature amount map F9 output from the ninth layer 39 is an image obtained by extracting the large vessel occlusion region in the CT image G0.

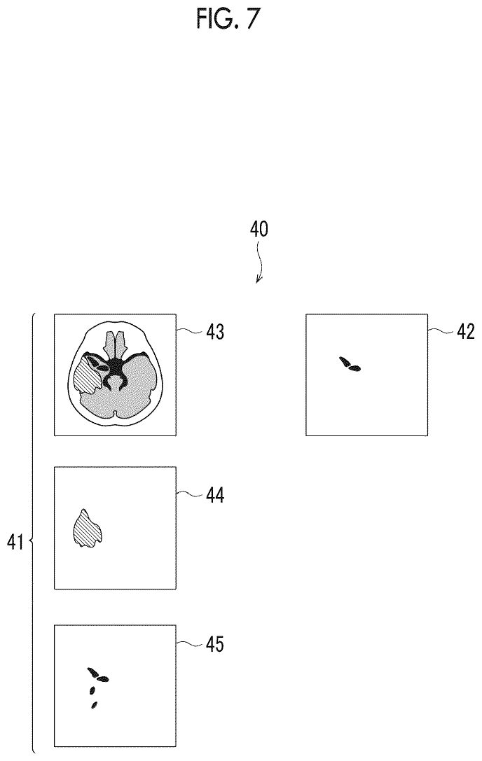

FIG. 7 is a diagram showing training data for training the U-Net corresponding to the third discriminative model 22C in the first embodiment. As shown in FIG. 7, training data 40 consists of input data 41 and correct answer data 42. The input data 41 consists of a non-contrast CT image 43, a mask image 44 representing the infarction region in the non-contrast CT image 43, and a mask image 45 representing the large vessel occlusion region candidate in the non-contrast CT image 43. The correct answer data 42 is a mask image representing the large vessel occlusion region in the non-contrast CT image 43. It should be noted that the mask image 45 is derived by inputting the non-contrast CT image 43 to the second discriminative model 22B.

In the present embodiment, a large amount of the training data 40 is stored in the image storage server 3, and the training data 40 is acquired from the image storage server 3 by the information acquisition unit 21 and is used for training the U-Net by the learning unit 23.

The learning unit 23 inputs the non-contrast CT image 43, the mask image 44, and the mask image 45, which are the input data 41, to the U-Net, and causes the U-Net to output the image representing the large vessel occlusion region in the non-contrast CT image 43. Specifically, the learning unit 23 causes the U-Net to extract the HAS in the non-contrast CT image 43 and to output a mask image in which a part of the HAS is masked. The learning unit 23 derives a difference between the output image and the correct answer data 42 as a loss, and learns the weight of the connection of each layer in the U-Net and a coefficient of kernel such that the loss is small. It should be noted that, in a case of the learning, a perturbation may be added to the mask images 44 and 45. As the perturbation, for example, morphology processing may be added to the mask with a random probability, or the mask may be subjected to zero padding. By adding the perturbation to the mask images 44 and 45, it is possible to handle a pattern observed in a case of the cerebral infarction in a hyperacute phase in which only the thrombus appears on the image without a remarkable infarction region, and it is further possible to prevent the third discriminative model 22C being excessively dependent on the input mask image in a case of the discrimination.

Then, the learning unit 23 repeatedly performs the learning until the loss is equal to or less than a predetermined threshold value. As a result, the third discriminative model 22C is constructed, the third discriminative model 22C extracting the large vessel occlusion region included in the CT image G0 as the third information, to output a mask image H0 representing the large vessel occlusion region in the CT image G0 in a case in which the non-contrast CT image G0, the mask image M0 representing the infarction region in the CT image G0, and the mask image M1 representing the large vessel occlusion region candidate in the CT image G0 are input. It should be noted that the learning unit 23 may construct the third discriminative model 22C by repeatedly performing the learning a predetermined number of times.

It should be noted that the configuration of the U-Net constituting the third discriminative model 22C is not limited to that shown in FIG. 5. For example, in the U-Net shown in FIG. 5, the inversion feature amount map F3A is derived from the feature amount map F3 output from the third layer 33, but the inversion feature amount map may be used in any layer in the U-Net. Moreover, the number of convolutional layers of each layer in the U-Net is not limited to that shown in FIG. 5.

It should be noted that, in the first embodiment, both the second discriminative model 22B and the third discriminative model 22C derive the large vessel occlusion region from the CT image G0. However, the third discriminative model 22C can derive the large vessel occlusion region with higher accuracy than the second discriminative model 22B because the third discriminative model 22C uses the infarction region and the large vessel occlusion region candidate. Therefore, in the present embodiment, the large vessel occlusion region derived by the second discriminative model 22B is used as the large vessel occlusion region candidate. It should be noted that the large vessel occlusion region candidate derived by the second discriminative model 22B may match the large vessel occlusion region derived by the third discriminative model 22C.

The quantitative value derivation unit 24 derives a quantitative value for at least one of the infarction region or the large vessel occlusion region derived by the information derivation unit 22. The quantitative value is an example of quantitative information in the present disclosure. In the present embodiment, it is assumed that the quantitative value derivation unit 24 derives the quantitative values of both the infarction region and the large vessel occlusion region, but the quantitative value of any one of the infarction region or the large vessel occlusion region may be derived. Since the CT image G0 is the three-dimensional image, the quantitative value derivation unit 24 may derive a volume of the infarction region, a volume of the large vessel occlusion region, and a length of the large vessel occlusion region as the quantitative values. Moreover, the quantitative value derivation unit 24 may derive a score of ASPECTS as the quantitative value.

The “ASPECTS” is an abbreviation for alberta stroke program early CT score, and is a scoring method in which an early CT sign of a simple CT is quantified for the cerebral infarction in a middle cerebral artery region. Specifically, the ASPECTS is a method in which, in a case in which the medical image is the CT image, the middle cerebral artery region is classified into 10 regions in two representative cross sections (basal ganglia level and radiation coronary level), the presence or absence of early ischemic change for each region is evaluated, and a positive part is scored by a point-deduction method. In the ASPECTS, an area of the infarction region is larger as the score is lower. The quantitative value derivation unit 24 need only derive the score depending on whether or not the infarction region is included in the 10 regions described above.

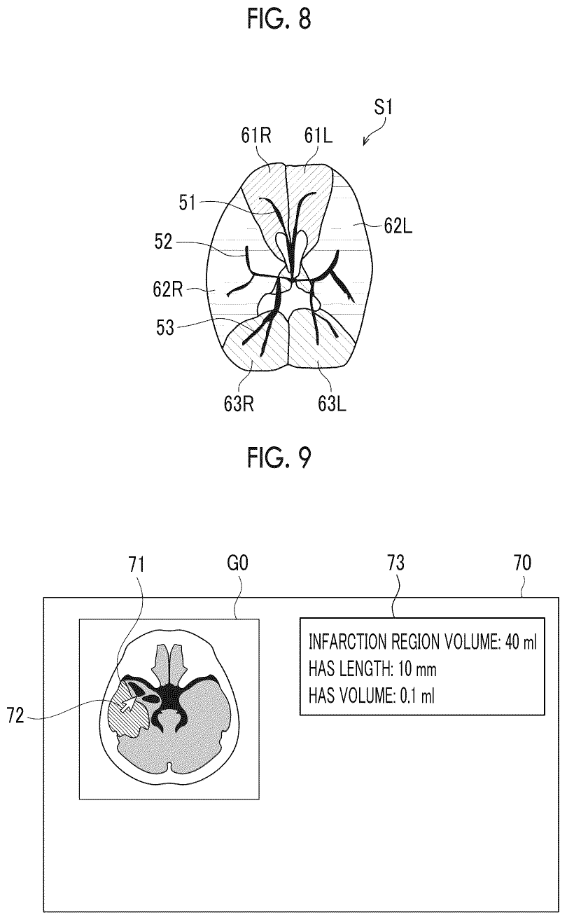

Moreover, the quantitative value derivation unit 24 may specify a dominant region of the occluded blood vessel based on the large vessel occlusion region, and derive an overlapping amount (volume) between the dominant region and the infarction region as the quantitative value. FIG. 8 is a diagram showing an artery and a dominant region in the brain. It should be noted that FIG. 8 shows a slice image SI on a certain tomographic plane of the CT image G0. As shown in FIG. 8, the brain includes an anterior cerebral artery (ACA) 51, a middle cerebral artery (MCA) 52, and a posterior cerebral artery (PCA) 53. Moreover, although not shown, an internal carotid artery (ICA) is also included. The brain is divided into middle cerebral artery dominant regions 62L and 62R, left and right anterior cerebral artery dominant regions 61L and 61R, and posterior cerebral artery dominant regions 63L and 63R in which the blood flows are dominated by the anterior cerebral artery 51, the middle cerebral artery 52, and the posterior cerebral artery 53, respectively. It should be noted that, in FIG. 8, a right side on the paper surface is a region on a left brain side of the brain.

It should be noted that the dominant region need only be specified by the registration of the CT image G0 with a prepared standard brain image in which the dominant region is specified.

The quantitative value derivation unit 24 specifies the artery in which the large vessel occlusion region is present, and specifies the dominant region by the specified artery of the brain. For example, in a case in which the large vessel occlusion region is present in the left anterior cerebral artery, the dominant region is specified as the anterior cerebral artery dominant region 61L. Here, the infarction region is generated downstream of the part in which the thrombus is present in the artery. Therefore, the infarction region is present in the anterior cerebral artery dominant region 61L. Therefore, the quantitative value derivation unit 24 need only derive the volume of the infarction region with respect to the volume of the anterior cerebral artery dominant region 61L in the CT image G0 as the quantitative value.

The display control unit 25 displays the CT image G0 of the patient and the quantitative value on the display 14. FIG. 9 is a diagram showing a display screen. As shown in FIG. 9, the slice image included in the CT image G0 of the patient is displayed on a display screen 70 in a switchable manner based on an operation of the input device 15. Moreover, a mask 71 of the infarction region is superimposed and displayed on the CT image G0. Moreover, an arrow-shaped mark 72 indicating the large vessel occlusion region is also superimposed and displayed. Moreover, on the right side of the CT image G0, a quantitative value 73 derived by the quantitative value derivation unit 24 is displayed. Specifically, the volume of the infarction region (40 ml), the length of the large vessel occlusion region (length of HAS: 10 mm), and the volume of the large vessel occlusion region (volume of HAS: 0.1 ml) are displayed. It should be noted that the large vessel occlusion region candidate may be displayed in addition to the large vessel occlusion region.

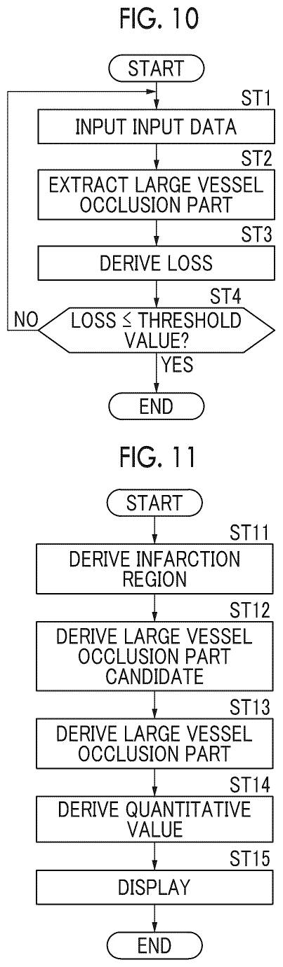

Next, processing performed in the first embodiment will be described. FIG. 10 is a flowchart showing learning processing performed in the first embodiment. It should be noted that it is assumed that the training data is acquired from the image storage server 3 and stored in the storage 13. First, the learning unit 23 inputs the input data 41 included in the training data 40 to the U-Net (step ST1), and causes the U-Net to extract the large vessel occlusion region (step ST2). Then, the learning unit 23 derives the loss from the extracted large vessel occlusion region and the correct answer data 42 (step ST3), and determines whether or not the loss is equal to or less than the predetermined threshold value (step ST4).

In a case in which a negative determination is made in step ST4, the processing returns to step ST1, and the learning unit 23 repeats the processing of step ST1 to step ST4. In a case in which a positive determination is made in step ST4, the processing ends. As a result, the third discriminative model 22C is constructed.

FIG. 11 is a flowchart showing information processing performed in the first embodiment. It should be noted that it is assumed that the non-contrast CT image G0 which is the processing target is acquired from the image storage server 3 and stored in the storage 13. First, the information derivation unit 22 derives the infarction region in the CT image G0 by using the first discriminative model 22A (step ST11). The information derivation unit 22 derives the large vessel occlusion region candidate in the CT image G0 by using the second discriminative model 22B (step ST12). Further, the information derivation unit 22 derives the large vessel occlusion region in the CT image G0 based on the CT image G0, the mask image M0 representing the infarction region in the CT image G0, and the mask image M1 representing the large vessel occlusion region candidate in the CT image G0, by using the third discriminative model 22C (step ST13).

Then, the quantitative value derivation unit 24 derives the quantitative value based on the information on the infarction region and the large vessel occlusion region (step ST14). Then, the display control unit 25 displays the CT image G0 and the quantitative value (step ST15), and ends the processing.

In this way, in the first embodiment, the large vessel occlusion region in the CT image G0 is derived based on the non-contrast CT image G0 of the head of the patient, the infarction region in the CT image G0, and the large vessel occlusion region candidate in the CT image. As a result, since the infarction region can be considered, the large vessel occlusion region can be accurately specified in the CT image G0.

Here, a brain disease, such as the cerebral infarction, is rarely developed simultaneously in both the left brain and the right brain. Therefore, by using the inversion feature amount map F3A in which the feature amount map F3 is inverted with respect to the midline CO of the brain, it is possible to specify the large vessel occlusion region while comparing the features of the left and right brains. As a result, the large vessel occlusion region can be specified with high accuracy.

Moreover, by displaying the quantitative value, a doctor can easily decide the treatment policy based on the quantitative value. For example, by displaying the volume or the length of the large vessel occlusion region, it is easy to decide a type or a length of a device used in the application of thrombectomy treatment method.

Next, a second embodiment of the present disclosure will be described. It should be noted that a configuration of an information processing apparatus in the second embodiment is the same as the configuration of the information processing apparatus in the first embodiment, only the processing to be performed is different, and thus the detailed description of the apparatus will be omitted.

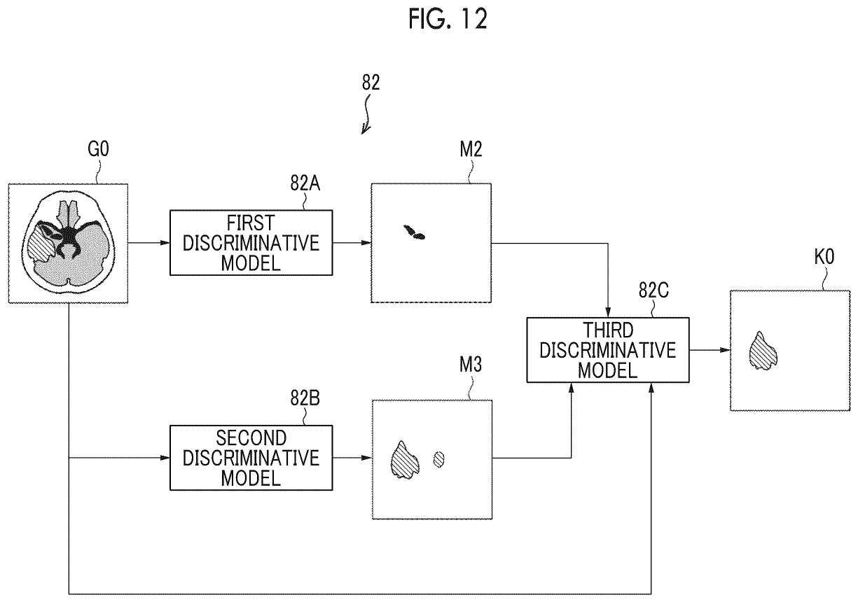

FIG. 12 is a schematic block diagram showing a configuration of an information derivation unit in the second embodiment. As shown in FIG. 12, an information derivation unit 82 according to the second embodiment includes a first discriminative model 82A, a second discriminative model 82B, and a third discriminative model 82C. The first discriminative model 82A in the second embodiment is constructed by training the CNN through machine learning to extract the large vessel occlusion region from the CT image G0 as the first information. For the construction of the first discriminative model 82A, for example, the method disclosed in JP2020-054580A can be used. Specifically, the first discriminative model 82A can be constructed by training the CNN through machine learning using, as the training data, the large vessel occlusion region in the non-contrast CT image and the non-contrast CT image of the head.

The second discriminative model 82B in the second embodiment is constructed by training the CNN through machine learning to extract the candidate of the infarction region, as the second information, from the CT image G0. For the construction of the second discriminative model 82B, for example, the method disclosed in JP2020-054580A can be used. Specifically, the second discriminative model 82B can be constructed by training the CNN through machine learning using, as the training data, the non-contrast CT image of the head and the infarction region in the non-contrast CT image. It should be noted that, in the second embodiment, both the second discriminative model 82B and the third discriminative model 82C extract the infarction region from the CT image G0, but the infarction region extracted by the second discriminative model 82B is used as an infarction region candidate.

The third discriminative model 82C in the second embodiment is constructed by training the U-Net through machine learning using a large amount of the training data to extract the infarction region from the CT image G0 as the third information based on the CT image G0, a mask image M2 representing the large vessel occlusion region in the CT image G0, and a mask image M3 representing the infarction region candidate in the CT image G0. It should be noted that the configuration of the U-Net is the same as that of the first embodiment, and thus the detailed description thereof will be omitted here.

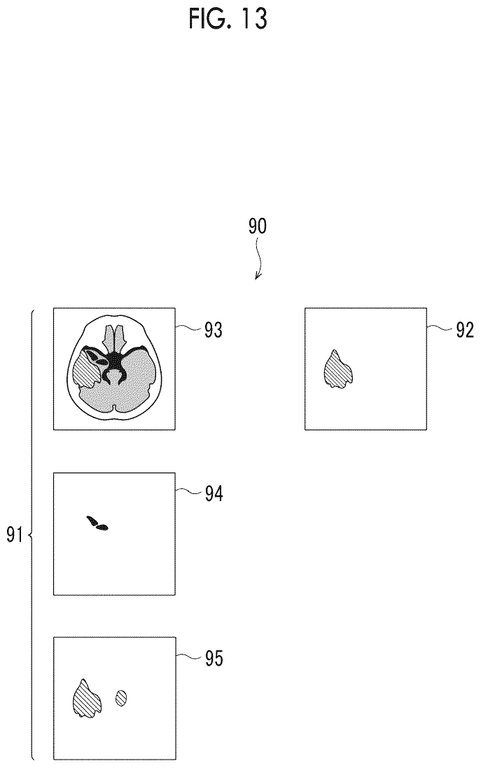

FIG. 13 is a diagram showing training data for training the U-Net corresponding to the third discriminative model 82C in the second embodiment. As shown in FIG. 13, training data 90 consists of input data 91 and correct answer data 92. The input data 91 consists of a non-contrast CT image 93, a mask image 94 representing the large vessel occlusion region in the non-contrast CT image 93, and a mask image 95 representing the infarction region candidate in the non-contrast CT image 93. The correct answer data 92 is a mask image representing the infarction region in the non-contrast CT image 93. It should be noted that the mask image 95 is derived by inputting the non-contrast CT image 93 to the second discriminative model 82B.

In the second embodiment, the learning unit 23 constructs the third discriminative model 82C by training the U-Net using a large amount of the training data 90 shown in FIG. 13. As a result, the third discriminative model 82C in the second embodiment extracts the infarction region in the CT image G0 to output the mask image KO representing the infarction region in a case in which the CT image G0, the mask image M2 representing the large vessel occlusion region, and the mask image M3 representing the cerebral infarction region candidate are input. It should be noted that, in the second embodiment, the third discriminative model 82C may extract the infarction region further based on the information on the symmetrical regions with respect to the midline of the brain in at least the CT image G0 out of the CT image G0, the mask image M2, and the mask image M3.

It should be noted that, in the second embodiment, both the second discriminative model 82B and the third discriminative model 82C derive the infarction region from the CT image G0. However, the third discriminative model 82C can derive the infarction region with higher accuracy than the second discriminative model 82B because the third discriminative model 82C uses the large vessel occlusion region and the infarction region candidate. Therefore, in the present embodiment, the infarction region derived by the second discriminative model 82B is the infarction region candidate. It should be noted that the infarction region candidate derived by the second discriminative model 82B may match the infarction region derived by the third discriminative model 82C.

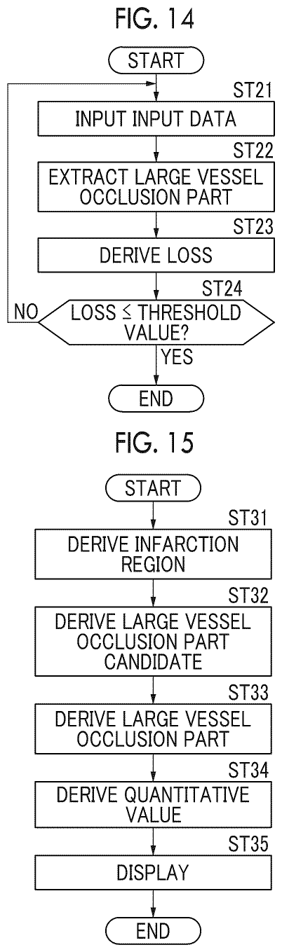

Next, processing performed in the second embodiment will be described. FIG. 14 is a flowchart showing learning processing performed in the second embodiment. It should be noted that it is assumed that the training data is acquired from the image storage server 3 and stored in the storage 13. First, the learning unit 23 inputs the input data 91 included in the training data 90 to the U-Net (step ST21), and causes the U-Net to extract the infarction region (step ST22). Then, the learning unit 23 derives the loss from the extracted infarction region and the correct answer data 92 (step ST23), and determines whether or not the loss is equal to or less than the predetermined threshold value (step ST24).

In a case in which a negative determination is made in step ST24, the processing returns to step ST21, and the learning unit 23 repeats the processing of step ST21 to step ST24. In a case in which a positive determination is made in step ST24, the processing ends. As a result, the third discriminative model 82C is constructed.

FIG. 15 is a flowchart showing information processing performed in the second embodiment. It should be noted that it is assumed that the non-contrast CT image G0 which is the processing target is acquired from the image storage server 3 and stored in the storage 13. First, the information derivation unit 82 derives the large vessel occlusion region in the CT image G0 by using the first discriminative model 82A (step ST31). The information derivation unit 82 derives the infarction region candidate in the CT image G0 by using the second discriminative model 82B (step ST32). Further, the information derivation unit 82 derives the infarction region in the CT image G0 based on the CT image G0, the mask image M2 representing the large vessel occlusion region in the CT image G0, and the mask image M3 representing the infarction region candidate in the CT image G0, by using the third discriminative model 82C (step ST33).

Then, the quantitative value derivation unit 24 derives the quantitative value based on the information on the infarction region and the large vessel occlusion region (step ST34). Then, the display control unit 25 displays the CT image G0 and the quantitative value (step ST35), and ends the processing.

In this way, in the second embodiment, the infarction region in the CT image G0 is derived based on the non-contrast CT image G0 of the head of the patient, the large vessel occlusion region in the CT image G0, and the infarction region candidate in the CT image. As a result, since the large vessel occlusion region can be considered, the infarction region can be accurately specified in the CT image G0.

Next, a third embodiment of the present disclosure will be described. It should be noted that a configuration of an information processing apparatus in the third embodiment is the same as the configuration of the information processing apparatus in the first embodiment, only the processing to be performed is different, and thus the detailed description of the apparatus will be omitted.

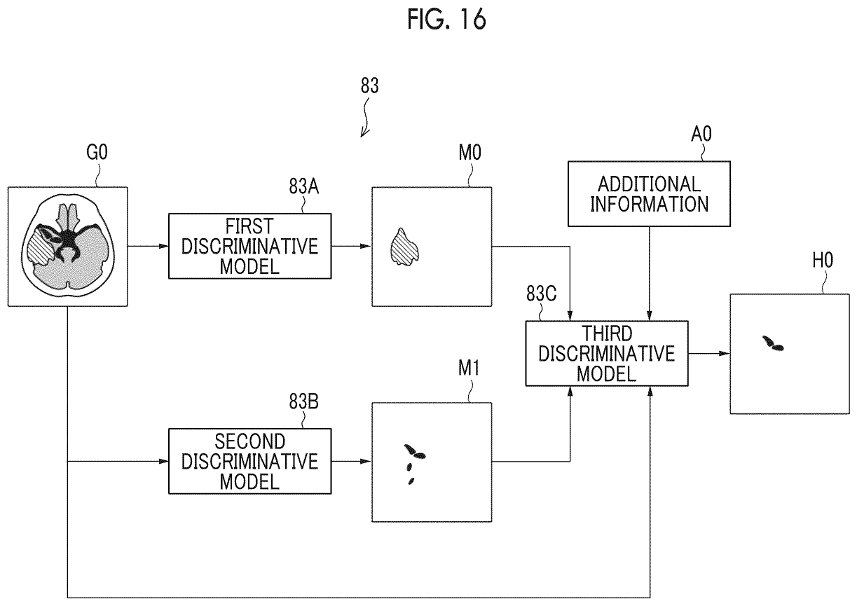

FIG. 16 is a schematic block diagram showing a configuration of an information derivation unit in the third embodiment. As shown in FIG. 16, an information derivation unit 83 according to the second embodiment includes a first discriminative model 83A, a second discriminative model 83B, and a third discriminative model 83C. Similarly to the first discriminative model 22A in the first embodiment, the first discriminative model 83A in the third embodiment is constructed by training the CNN through machine learning to extract the infarction region from the CT image G0 as the first information. The second discriminative model 83B in the third embodiment is constructed by training the CNN through machine learning to extract the large vessel occlusion region candidate as the second information from the CT image G0, in the same manner as the second discriminative model 22B in the first embodiment.

The third discriminative model 83C in the third embodiment is constructed by training the U-Net through machine learning using a large amount of the training data to extract the large vessel occlusion region from the CT image G0 as third information based on the CT image G0, the mask image M0 representing the infarction region in the CT image G0, the mask image M1 representing the large vessel occlusion region candidate in the CT image G0, and at least one information (hereinafter, referred to as additional information A0) of the information representing the anatomical region of the brain or the clinical information. It should be noted that the configuration of the U-Net is the same as that of the first embodiment, and thus the detailed description thereof will be omitted here.

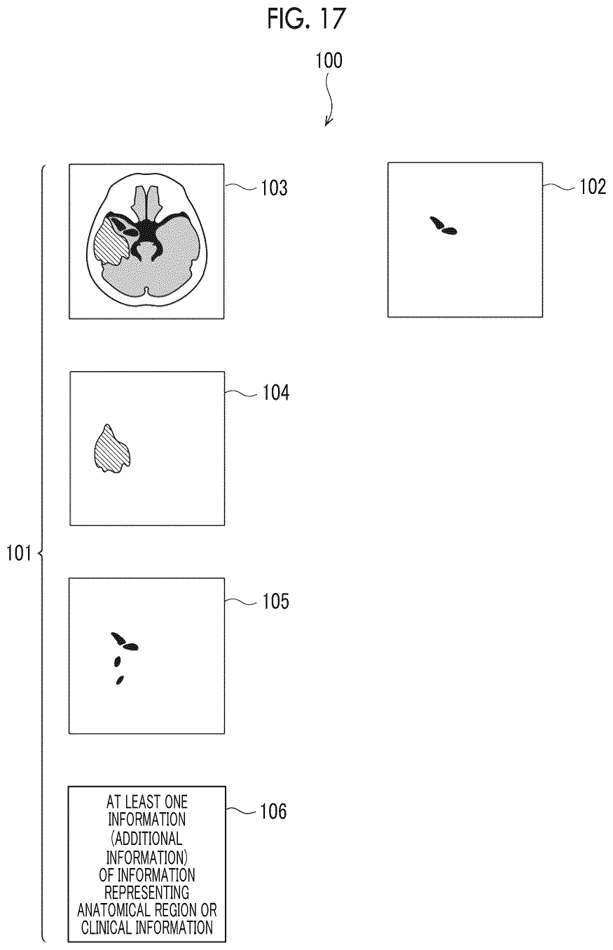

FIG. 17 is a diagram showing training data for training the U-Net corresponding to the third discriminative model 83C in the third embodiment. As shown in FIG. 17, training data 100 consists of input data 101 and correct answer data 102. The input data 101 consists of the non-contrast CT image 103, a mask image 104 representing an infarction region in the non-contrast CT image 103, a mask image 105 representing a large vessel occlusion region candidate in the non-contrast CT image 103, and at least one information (referred to as additional information) 106 of the information representing the anatomical region or the clinical information. The correct answer data 102 is a mask image representing the large vessel occlusion region in the non-contrast CT image 103. It should be noted that the mask image 105 is derived by inputting the non-contrast CT image 103 to the second discriminative model 83B.

Here, as the information representing the anatomical region, for example, a mask image of the blood vessel dominant region in which the infarction region is present in the non-contrast CT image 103 can be used. Moreover, the mask image of the region of the ASPECTS in which the infarction region is present in the non-contrast CT image 103 can be used as the information representing the anatomical region. As the clinical information, a score of the ASPECTS for the non-contrast CT image 103 and a national institutes of health stroke scale (NIHSS) for the patient from whom the non-contrast CT image 103 is acquired can be used. The NIHSS is one of the most widely used evaluation methods in the world as an evaluation scale for the severity of stroke neurology.

In the third embodiment, the learning unit 23 constructs the third discriminative model 83C by training the U-Net using a large amount of the training data 100 shown in FIG. 17. As a result, in the third embodiment, the third discriminative model 83C extracts the large vessel occlusion region from the CT image G0 to output the mask image H0 representing the large vessel occlusion region in a case in which the CT image G0, the mask image M0 representing the infarction region, the mask image M1 representing the large vessel occlusion region candidate, and the additional information A0 are input.

It should be noted that the learning processing in the third embodiment is different from that in the first embodiment only in that the additional information A0 is used, and thus the detailed description of the learning processing will be omitted. The information processing in the third embodiment is different from the information processing in the first embodiment only in that the information input to the third discriminative model 83C includes the additional information A0 of the patient in addition to the CT image G0 and the mask image representing the infarction region, and thus the detailed description of the information processing will be omitted.

In this way, in the third embodiment, the large vessel occlusion region in the CT image G0 is derived based on the additional information A0 in addition to the non-contrast CT image G0 of the head of the patient, the infarction region in the CT image G0, and the large vessel occlusion region candidate in the CT image. As a result, since the additional information can be also taken into consideration in addition to the infarction region, the large vessel occlusion region can be specified with higher accuracy in the CT image G0.

It should be noted that, in the third embodiment, the third discriminative model 83C is constructed to extract the large vessel occlusion region in the CT image G0 in a case in which the CT image G0, the mask image M0 representing the infarction region, the mask image M1 representing the large vessel occlusion region candidate, and the additional information A0 are input, but the present disclosure is not limited to this. The third discriminative model 83C may be constructed to extract the infarction region in the CT image G0 in a case in which the CT image G0, the mask image representing the large vessel occlusion region, the mask image representing the infarction region candidate, and the additional information are input.

In addition, in each of the above-described embodiments, the third discriminative model derives the second information (that is, the infarction region or the large vessel occlusion region) by using the information on the symmetrical regions with respect to the midline of the brain in the CT image G0, the first information, the second information, the information representing the anatomical region of the brain, and the clinical information, but the present disclosure is not limited to this. The second discriminative model may be constructed to derive the third information without using the information on the symmetrical regions with respect to the midline of the brain in the CT image G0, the first information, the second information, the information representing the anatomical region of the brain, and the clinical information.

In the first embodiment, the third information representing the large vessel occlusion region is derived based on the first information representing the infarction region and the second information representing the large vessel occlusion region candidate, but the present disclosure is not limited to this. The third information representing the large vessel occlusion region may be derived based on the information representing the anatomical region of the brain and the second information representing the large vessel occlusion region candidate, instead of or in addition to the first information. The third information representing the large vessel occlusion region may be derived based on the clinical information and the second information representing the large vessel occlusion region candidate, instead of or in addition to the first information. The third information representing the large vessel occlusion region may be derived based on the information representing the anatomical region of the brain, the clinical information, and the second information representing the large vessel occlusion region candidate, instead of the first information.

In the second embodiment, the third information representing the infarction region is derived based on the first information representing the large vessel occlusion region and the second information representing the infarction region candidate, but the present disclosure is not limited to this. The third information representing the infarction region may be derived based on the information representing the anatomical region of the brain and the second information representing the infarction region candidate, instead of or in addition to the first information. In addition, the third information representing the infarction region may be derived based on the clinical information and the second information representing the infarction region candidate, instead of or in addition to the first information. In addition, the third information representing the infarction region may be derived based on the information representing the anatomical region of the brain, the clinical information, and the second information representing the infarction region candidate, instead of the first information.

In each of the above-described embodiments, the third discriminative model is constructed by using U-Net, but the present disclosure is not limited to this. The third discriminative model may be constructed by using a convolutional neural network other than the U-Net.

In the embodiment described above, the third information is derived by inputting the CT image G0 to the third discriminative model, but the present disclosure is not limited to this. The third discriminative model may be constructed to derive the third information without using the CT image G0. In this case, the third discriminative model is constructed by being trained without using the CT image as the input data of the training data.

Moreover, in each of the embodiments described above, in the first discriminative models 22A, 82A, and 83A of the information derivation units 22, 82, and 83, the first information (that is, the infarction region or the large vessel occlusion region) is derived from the CT image G0 by using the CNN, but the present disclosure is not limited to this. The information derivation unit may acquire the mask image generated by a doctor by interpreting the CT image G0 to specify the infarction region or the large vessel occlusion region as the first information without using the first discriminative model, and derive the third information.

In each of the above-described embodiments, the second discriminative models 22B, 82B, and 83B of the information derivation units 22, 82, and 83 use the CNN to derive the second information (that is, the infarction region candidate or the large vessel occlusion region candidate) from the CT image G0, but the present disclosure is not limited to this. The information derivation unit may derive the third information by acquiring the mask image generated by the doctor performing the interpretation on the CT image G0 to specify the infarction region candidate or the large vessel occlusion region candidate as the second information without using the second discriminative model.

Moreover, in each of the embodiments described above, the information derivation units 22, 82, and 83 derive the infarction region and the large vessel occlusion region, but the present disclosure is not limited to this. A bounding box that surrounds the infarction region and the large vessel occlusion region may be derived.

Moreover, in the embodiments described above, for example, various processors shown below can be used as the hardware structures of processing units that execute various processing, such as the information acquisition unit 21, the information derivation unit 22, the learning unit 23, the quantitative value derivation unit 24, and the display control unit 25 in the information processing apparatus 1. As described above, in addition to the CPU which is a general-purpose processor that executes the software (program) to function as the various processing units described above, the various processors include a programmable logic device (PLD), which is a processor of which a circuit configuration can be changed after manufacturing, such as a field programmable gate array (FPGA), a dedicated electric circuit, which is a processor having a circuit configuration exclusively designed to execute specific processing, such as an application specific integrated circuit (ASIC), and the like.

One processing unit may be configured by one of these various processors, or may be configured by a combination of two or more processors of the same type or different types (for example, a combination of a plurality of FPGAs or a combination of the CPU and the FPGA). Moreover, a plurality of processing units may be configured by one processor. A first example of the configuration in which the plurality of processing units are configured by one processor is a form in which one processor is configured by a combination of one or more CPUs and software and the processor functions as the plurality of processing units as represented by the computer, such as a client and a server. A second example thereof is a form in which a processor that realizes the function of the entire system including the plurality of processing units by one integrated circuit (IC) chip is used, as represented by a system-on-chip (SoC) or the like. As described above, as the hardware structures, the various processing units are configured by using one or more of the various processors described above.

Further, as the hardware structures of these various processors, more specifically, it is possible to use an electric circuit (circuitry) in which circuit elements, such as semiconductor elements, are combined.

What is claimed is:

1. An information processing apparatus comprising:
at least one processor,
wherein the processor
acquires at least one of first information representing any one of an infarction region or a large vessel occlusion region in a non-contrast CT image of a head of a patient, information representing an anatomical region of a brain, or clinical information,
acquires second information representing a candidate of the other of the infarction region or the large vessel occlusion region in the non-contrast CT image, and
derives third information representing the other of the infarction region or the large vessel occlusion region in the non-contrast CT image based on at least one of the first information, the information representing the anatomical region of the brain, or the clinical information, and the second information.

2. The information processing apparatus according to claim 1,
wherein the processor
further acquires the non-contrast CT image, and
derives the third information further based on the non-contrast CT image.

3. The information processing apparatus according to claim 2,
wherein the processor derives the third information by using a discriminative model that has been trained to output the third information in a case in which at least one of the first information, the information representing the anatomical region of the brain, or the clinical information, the non-contrast CT image, and the second information are input.

4. The information processing apparatus according to claim 2,
wherein the processor derives the third information further based on information on symmetrical regions with respect to a midline of the brain in at least the non-contrast CT image out of the first information, the non-contrast CT image, and the second information.

5. The information processing apparatus according to claim 4,
wherein the information on the symmetrical regions is inversion information obtained by inverting at least the non-contrast CT image out of the first information, the non-contrast CT image, and the second information, with respect to the midline of the brain.

6. The information processing apparatus according to claim 1,
wherein the processor acquires the first information by extracting any one of the infarction region or the large vessel occlusion region from the non-contrast CT image, and acquires the second information by extracting the candidate of the other of the infarction region or the large vessel occlusion region from the non-contrast CT image.

7. The information processing apparatus according to claim 1,
wherein the processor
derives quantitative information for at least one of the first information, the second information, or the third information, and
displays the quantitative information.

8. A learning device comprising:
at least one processor,
wherein the processor
acquires i) a non-contrast CT image of a head of a patient with cerebral infarction, ii) at least one of first information representing any one of an infarction region or a large vessel occlusion region in the non-contrast CT image, information representing an anatomical region of a brain, or clinical information, and iii) training data including input data consisting of second information representing a candidate of the other of the infarction region or the large vessel occlusion region in the non-contrast CT image, and correct answer data consisting of third information representing the other of the infarction region and the large vessel occlusion region in the non-contrast CT image, and
trains a neural network through machine learning using the training data to construct a discriminative model that outputs the third information in a case in which at least one of the first information, the information representing the anatomical region of the brain, or the clinical information, the non-contrast CT image, and the second information are input.

9. A discriminative model that, in a case in which i) a non-contrast CT image of a head of a patient, ii) at least one of first information representing any one of an infarction region or a large vessel occlusion region in the non-contrast CT image, information representing an anatomical region of a brain, or clinical information, and iii) second information representing a candidate of the other of the infarction region or the large vessel occlusion region in the non-contrast CT image are input, outputs third information representing the other of the infarction region or the large vessel occlusion region in the non-contrast CT image.

10. An information processing method comprising:
acquiring at least one of first information representing any one of an infarction region or a large vessel occlusion region in a non-contrast CT image of a head of a patient, information representing an anatomical region of a brain, or clinical information;
acquiring second information representing a candidate of the other of the infarction region or the large vessel occlusion region in the non-contrast CT image; and
deriving third information representing the other of the infarction region or the large vessel occlusion region in the non-contrast CT image based on at least one of the first information, the information representing the anatomical region of the brain, or the clinical information, and the second information.

11. A learning method comprising:
acquiring i) a non-contrast CT image of a head of a patient with cerebral infarction, ii) at least one of first information representing any one of an infarction region or a large vessel occlusion region in the non-contrast CT image, information representing an anatomical region of a brain, or clinical information, and iii) training data including input data consisting of second information representing a candidate of the other of the infarction region or the large vessel occlusion region in the non-contrast CT image, and correct answer data consisting of third information representing the other of the infarction region and the large vessel occlusion region in the non-contrast CT image; and
training a neural network through machine learning using the training data to construct a discriminative model that outputs the third information in a case in which at least one of the first information, the information representing the anatomical region of the brain, or the clinical information, the non-contrast CT image, and the second information are input.

12. A non-transitory computer-readable storage medium that stores an information processing program causing a computer to execute:
a procedure of acquiring at least one of first information representing any one of an infarction region or a large vessel occlusion region in a non-contrast CT image of a head of a patient, information representing an anatomical region of a brain, or clinical information;
a procedure of acquiring second information representing a candidate of the other of the infarction region or the large vessel occlusion region in the non-contrast CT image; and
a procedure of deriving third information representing the other of the infarction region or the large vessel occlusion region in the non-contrast CT image based on at least one of the first information, the information representing the anatomical region of the brain, or the clinical information, and the second information.

13. A non-transitory computer-readable storage medium that stores a learning program causing a computer to execute:
a procedure of acquiring i) a non-contrast CT image of a head of a patient with cerebral infarction, ii) at least one of first information representing any one of an infarction region or a large vessel occlusion region in the non-contrast CT image, information representing an anatomical region of a brain, or clinical information, and iii) training data including input data consisting of second information representing a candidate of the other of the infarction region or the large vessel occlusion region in the non-contrast CT image, and correct answer data consisting of third information representing the other of the infarction region and the large vessel occlusion region in the non-contrast CT image; and
a procedure of training a neural network through machine learning using the training data to construct a discriminative model that outputs the third information in a case in which at least one of the first information, the information representing the anatomical region of the brain, or the clinical information, the non-contrast CT image, and the second information are input.
